(19) United States
(12) Patent Application Publication
(10) Pub. No.: US 2023/0040593 A1
(43) Pub. Date: Feb. 9, 2023

(54) EXTRACORPOREAL CIRCULATION BLOOD PUMP AND METHOD THEREOF

(71) Applicant: SHANDONG UNIVERSITY, SHANDONG (CN)

(72) Inventors: Shuqin LIU, SHANDONG (CN); Zhongguo BIAN, SHANDONG (CN)

(73) Assignee: SHANDONG UNIVERSITY, SHANDONG (CN)

(21) Appl. No.: 17/786,541

(22) PCT Filed: Sep. 11, 2020
(86) PCT No.: PCT/CN2020/114872

§ 371 Date: Jun. 17, 2022

(30) Foreign Application Priority Data
Dec. 18, 2019 (CN) 201911310152.0

(51) Int. Cl. A61M 60/117; A61M 60/216; A61M 60/419; A61M 60/822; A61M 60/81; A61M 60/804

(52) U.S. Cl. CPC A61M 60/117; A61M 60/216; A61M 60/419; A61M 60/822; A61M 60/81; A61M 60/804

(57) ABSTRACT

An extracorporeal circulation blood pump and a method thereof are provided. A driving motor is driven to operate, and a rotating head rotates, thereby driving a rotator in a pump to rotate, an electromagnet is controlled to produce an axial upward attractive force on the rotator, so that the attraction force is matched with a coupling between driving permanent magnets and driven permanent magnets, and the rotator can rotate without contact in the axial direction. A first radial support permanent magnet and a second radial support permanent magnet are configured to interact with each other to generate a repulsive force, and the rotator can rotate without contact in the radial direction, achieving complete non-contact rotation with a pump housing, so as to enable a complex impeller to rotate without bearing support.

### TECHNICAL FIELD

The present disclosure relates to the technical field of extracorporeal circulation blood pumps, and specifically, to an extracorporeal circulation blood pump and a method thereof.

### DESCRIPTION OF RELATED ART

The description in this section merely provides background information related to the present disclosure and does not necessarily constitute the prior art.

A blood pump is the power part and the core part of an extracorporeal circulation system device. It mainly functions to replace the pulsation of the ventricle and recover blood lost during surgery, or perfuse blood after cardiac arrest. A centrifugal blood pump is a new type of extracorporeal circulation blood pump and is characterized by slight hemolysis, large pressure buffer, adjustable flow, high safety, and small volume. However, in the centrifugal blood pump currently used for extracorporeal circulation, a rotating impeller is still supported by a bearing, which will damage blood cells during operation, resulting in thrombosis.

### SUMMARY

To resolve the foregoing problem, the present disclosure provides an extracorporeal circulation blood pump and a method thereof. In the present disclosure, the rotation of an impeller can be ensured by the combination of permanent magnet and electromagnet without bearing support, which can effectively prevent thrombosis.

According to some embodiments, the present disclosure adopts the following technical solutions:

An extracorporeal circulation blood pump includes a pump holder, a pump body, a drive mechanism, and a control mechanism.

The pump holder has an accommodating cavity for carrying the pump body, and a plurality of first radial support permanent magnets are provided on an inner edge of the accommodating cavity.

The pump body includes a pump housing containing a rotator with a second radial support permanent magnet disposed on an outer circumference of a lower end in the rotator and a plurality of driven permanent magnets circumferentially distributed on the lower end in the rotator, and the first and second radial support permanent magnets are configured to interact with each other to generate a repulsive force, so that the rotator can rotate in a radial direction without contact.

The drive mechanism, disposed at a bottom portion of the accommodating cavity, includes a driving member and a rotating head, the driving member provides power to the blood pump through the rotating head, the rotating head includes a rotating disc connected to an output end of the driving member and a plurality of driving permanent magnets circumferentially disposed on the rotating disc, and a coupling between the driving permanent magnets and the driven permanent magnets is achieved by magnetic attraction, so that the rotator can rotate in an axial direction without contact.

The control mechanism includes an electromagnet disposed on an upper portion of the pump holder and a control portion, the electromagnet is provided to generate an axial upward attraction to the rotator in the pump, and a magnitude of the attraction is controlled by the control portion.

In an optional implementation, the pump holder includes the upper portion and a lower portion that are movably connected to each other and can be opened and closed freely, and the first radial support permanent magnets are disposed on a lower portion of the pump holder.

In an optional implementation, the upper portion of the pump holder includes a recess, an opening for accommodating the pump housing is provided in a middle portion of the recess, the recess can accommodate an upper surface of the pump body, and the electromagnet is circumferentially disposed at the opening on the upper portion of the pump holder.

In an optional implementation, the upper portion and the lower portion of the pump holder are connected by a rotating shaft on one side and are provided with a fastener on the other side.

Through the rotatably connection by the rotating shaft, the free opening and closing is achieved, which is convenient for taking and placing the pump body. The fastener is provided to ensure the sealing of the pump holder during the operation of the pump body.

In an optional implementation, the lower portion of the pump holder is provided with a stepped recess, the first radial support permanent magnets are disposed on one step of the stepped recess, and the drive mechanism is disposed at a bottom portion of the pump holder.

The stepped recess and the recess of the upper portion form the accommodating cavity for accommodating the pump body redundantly.

In an optional implementation, the drive mechanism includes a driving motor and the rotating head, the driving motor is fixed on the pump holder, and the rotating head is fixed on a top portion of a rotating shaft of the driving motor, a middle portion of the rotating disc of the rotating head is connected to the rotating shaft of the driving motor, and the driving permanent magnets are embedded circumferentially on the rotating disc.

In an optional implementation, the rotator includes a complex impeller and a permanent magnet assembly, the complex impeller is located on an upper portion of the rotator, and the permanent magnet assembly is disposed on a lower portion of the rotator.

As a further limitation, the complex impeller includes a magnetic conductor and an impeller body, the magnetic conductor is embedded in an upper cover of the impeller body, and the impeller body has a round hole in a center of the upper cover for the circulation of blood, and blood flows from an inlet of the pump housing, through the round hole and rotating blades, into a flow channel in the pump housing, and out of an outlet of the pump housing.

As a further limitation, the permanent magnet assembly includes the second radial support permanent magnet, the driven permanent magnets, and a sealed chamber, the sealed chamber is a cylindrical structure provided in a rotation center of the rotator and having a round hole, the second radial support permanent magnet is circumferentially embedded on an outer side of the sealed chamber, and the driven permanent magnets are circumferentially embedded on an inner side of the sealed chamber.

In an optional implementation, the control mechanism includes the electromagnet, a sensor, and the control portion, and the sensor is disposed on the upper portion of the pump holder and is configured to sense the rotation of the rotator in the pump housing and provide sensed information to the control portion.

As a further limitation, the control portion generates control information through analysis and calculation according to the sensed information from the sensor, and controls the electromagnet through a control circuit, so that the rotator rotates without contact at all with the pump housing under the combined action of the electromagnet, the driving permanent magnets, and the first and second radial support permanent magnets.

A method for operation of the blood pump based on the above includes the following steps. Activating the drive mechanism to rotate the rotating head, thereby driving the rotator in the pump body to rotate. Controlling the electromagnet, resulting in the axial upward attraction from the electromagnet to the rotator, so that the attraction matches with a coupling attraction between the driving permanent magnets and the driven permanent magnets, and enabling the rotator to rotate in the axial direction without contact. Configuring the first and second radial support permanent magnets to interact with each other to generate the repulsive force, enabling the rotator to rotate in the radial direction without contact, and leading to rotation without contact at all with the pump housing.

Compared with the prior art, the present disclosure has the following beneficial effects.

In the present disclosure, the impeller rotates without bearing support at all, greatly reducing the damage to blood cells and reducing the possibility of thrombosis.

In the present disclosure, the sensor senses the rotation of the rotator in the pump housing and provides sensed information to the control portion, and the control portion generates control information through analysis and calculation based on the sensed information and controls the electromagnet through a control circuit, so that the rotator rotates without contact at all with the pump housing under the combined action of the electromagnet, the driving permanent magnets, and the first and second radial support permanent magnets, which is convenient in control process and simple in control algorithm.

### BRIEF DESCRIPTION OF THE DRAWINGS

The accompanying drawings constituting a part of the present disclosure are used to provide further understanding of the present disclosure. Exemplary embodiments of the present disclosure and descriptions thereof are used to explain the present disclosure, and do not constitute an improper limitation to the present disclosure.

FIG. 1 is a schematic diagram of an overall structure according to the present disclosure;

FIG. 2 is a top view of a rotating head structure according to the present disclosure;

FIG. 3 is a side view of a rotator according to the present disclosure;

FIG. 4 is a top view of a permanent magnet assembly according to the present disclosure; and

FIG. 5 is a top view of an impeller body according to the present disclosure.

### DESCRIPTION OF THE EMBODIMENTS

The present disclosure is further described below with reference to the accompanying drawings and embodiments.

It should be noted that the following detailed descriptions are all exemplary and are intended to provide a further description of the present disclosure. Unless otherwise specified, all technical and scientific terms used herein have the same meaning as commonly understood by a person of ordinary skill in the art to which the present disclosure belongs.

It should be noted that terms used herein are only for describing specific implementations and are not intended to limit exemplary implementations according to the present disclosure. As used herein, the singular form is also intended to include the plural form unless the context clearly dictates otherwise. In addition, it should further be understood that, terms “comprise” and/or “include” used in this specification indicate that there are features, steps, operations, devices, components, and/or combinations thereof.

In the present disclosure, orientation or position relationships indicated by the terms such as “upper”, “lower”, “left”, “right” “front”, “rear”, “vertical”, “horizontal”, “side”, and “bottom” are based on orientation or position relationships shown in the accompanying drawings, and are merely relationship words that are determined for ease of describing the structural relationship between components or elements in the present disclosure, and are not intended to specifically refer to any component or element in the present disclosure. Therefore, such terms should not be construed as a limitation on the present disclosure.

In the present disclosure, terms such as “fixedly connected”, “interconnection”, and “connection” should be understood in a broad sense. The connection may be a fixing connection, an integral connection or a detachable connection; or the connection may be a direct connection, or an indirect connection by using an intermediary. Relevant scientific research or technical personnel in the art may determine the specific meanings of the foregoing terms in the present disclosure according to specific situations, and such terms should not be construed as a limitation on the present disclosure.

In order to resolve the problem of thrombosis as a result of blood cells damaged by using the centrifugal blood pump currently used for extracorporeal circulation during operation, this embodiment adopts the technical solution of combining permanent magnet with electromagnet to rotate an impeller without bearing support at all.

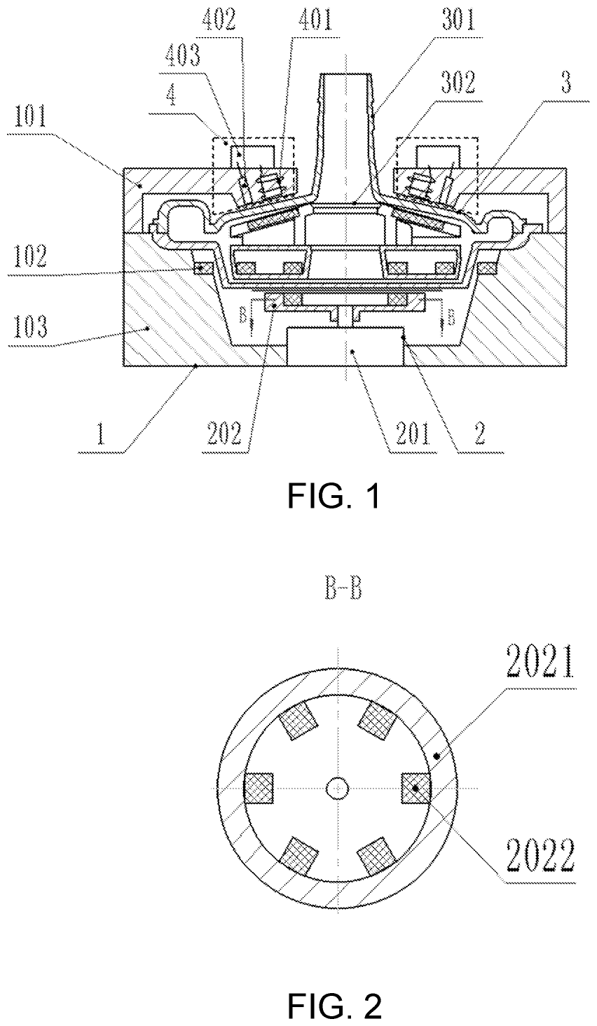

Specifically, as shown in FIG. 1, an extracorporeal circulation blood pump includes a pump holder 1, a drive portion 2, a pump 3, an electromagnet, and a control portion 4.

The pump holder 1 includes an upper portion 101, a first radial support permanent magnet 102, and a lower portion 103. The upper portion 101 and the lower portion 103 are connected by a rotating shaft to achieve free opening and closing of the upper portion 101 and facilitate taking and placing the pump 3. The first radial support permanent magnet 102 is disposed on the lower portion 103.

The upper portion 101 includes two parts that are connected by rotating shafts respectively and fixed by a fastener to the lower portion 103. The upper portion 101 is configured to fix the electromagnet and control portion 4 and allow an electromagnet 401 and a control sensor 402 to closely attached to the pump 3, so as to fully utilize the magnetic force of the electromagnet 401 and the sensitivity of the control sensor 402.

The first radial support permanent magnet 102 is a ring-shaped permanent magnet and interacts with a second radial support permanent magnet 30221 on an outer circumference of a lower portion 3022 in a rotator 302 in the pump 3 to generate a repulsive force, so that the rotator 302 can rotate in the radial direction without contact.

The lower portion 103 is designed with a stepped recess for supporting the pump 3, embedding the first radial support permanent magnet 102, and fixing the drive portion 2.

The drive portion 2 includes a driving motor 201 and a rotating head 202. The driving motor 201 is fixed on the pump holder 1. The rotating head 202 is fixed on the top portion of a rotating shaft of the driving motor 201.

The driving motor 201 provides power to the extracorporeal circulation blood pump through the rotating head 202.

As shown in FIG. 2, the rotating head 202 includes a rotating disc 2021 and driving permanent magnets 2022. The middle portion of the rotating disc 2021 is connected to the rotating shaft of the driving motor 201, and the driving permanent magnets 2022 are embedded circumferentially on the rotating disc 2021.

The driving permanent magnets 2022 include a number of permanent magnets embedded circumferentially on the rotating disc 2021. Correspondingly, driven permanent magnets 30222 on the rotator 302 of the pump 3 are also embedded circumferentially in a sealed chamber 30223. In this case, the coupling between the driving permanent magnets 2022 and the driven permanent magnets 30222 on the rotator 302 of the pump 3 is achieved by magnetic attraction. Due to the coupling, the driving motor 201 rotates to drive the rotating head 202 and further drive the rotator 302 in the pump 3 to rotate. An axial downward attraction acts with an axial upward attraction generated on the rotator 302 by the electromagnet 401 together, so that the rotator 302 can rotate in the axial direction without contact. In addition, the rotator 302 can rotate in the radial direction without contact, so that the impeller can rotate without bearing support at all.

The pump 3 includes a pump housing 301 and the rotator 302. The pump housing 301 is placed in the recess of the lower portion 103 of the pump holder 1, which can be easily separated from the pump holder. The rotator 302 is freely placed in the pump housing 301.

The pump housing 301 is provided with a cavity, a flow channel, an inlet, and an outlet.

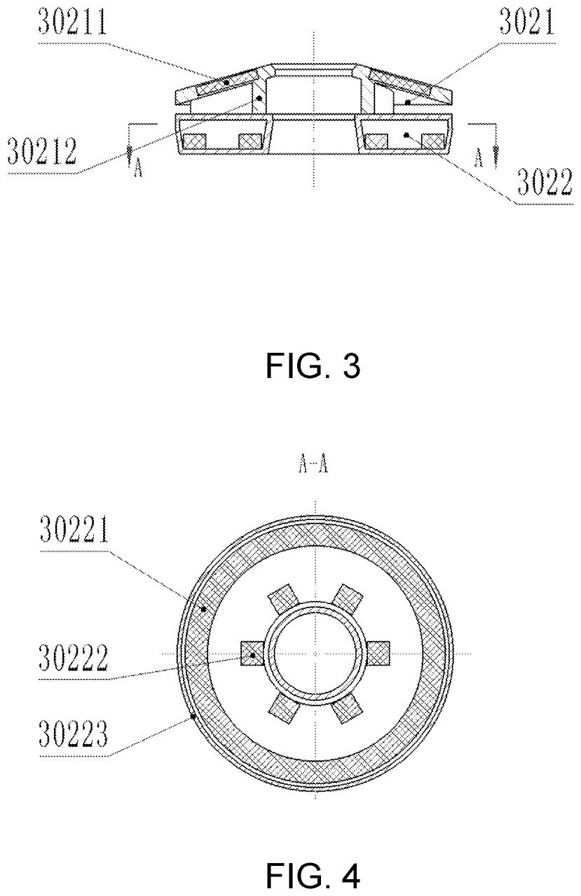

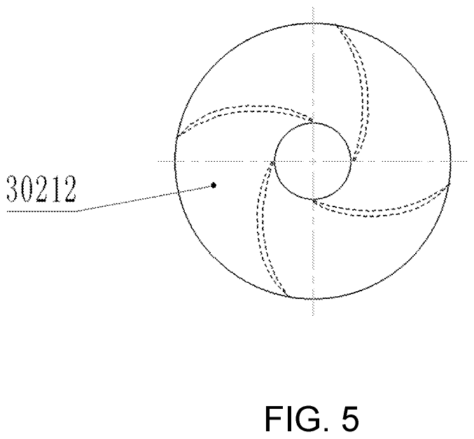

As shown in FIG. 3, the rotator 302 includes a complex impeller 3021 and a permanent magnet assembly 3022. The complex impeller 3021 is located on an upper portion of the rotator 302, and the permanent magnet assembly 3022 is located on a lower portion of the rotator 302.

The complex impeller 3021 includes a magnetic conductor 30211 and an impeller body 30212. The magnetic conductor 30211 is embedded in an upper cover of the impeller body 30212.

The magnetic conductor 30211 is configured for the sensor 402 to sense the rotator 302 and for the electromagnet 401 to generate attraction to the rotator 302.

The upper cover of the impeller body 30212 is provided with a round hole in a rotation center for the circulation of blood. Blood flows from the inlet of the pump housing 301, through the round hole provided in the rotation center of the impeller 3021 and through rotating blades, into the flow channel in the pump housing 301, and out of the outlet of the pump housing 301.

As shown in FIG. 4, the permanent magnet assembly 3022 includes the second radial support permanent magnet 30221, the driven permanent magnets 30222, and the sealed chamber 30223. The sealed chamber 30223 is a cylinder with the round hole provided in the rotation center. The second radial support permanent magnet 30221 is circumferentially embedded on an outer side of the sealed chamber 30223. The driven permanent magnets 30222 are circumferentially embedded on an inner side of the sealed chamber 30223.

The electromagnet and control portion 4 includes the electromagnet 401, the sensor 402, and a control portion 403. The control portion 403 may be a controller or a control circuit.

The electromagnet 401 is disposed on the upper portion of the pump holder and is provided to generate an axial upward attraction to the rotator 302 in the pump 3, and the magnitude of the attraction is controlled by the control portion 403. This axial upward attraction acts with the axial downward attraction generated to the rotator 302 by the driving permanent magnets 2022 and the radial repulsive force generated to the rotator 302 by the first radial support permanent magnet 102 together, so that the rotator 302 in the pump 3 rotates without bearing support at all to rotate the complex impeller 3021 without contact at all with the pump housing 301, thereby reducing the damage to blood cells caused by the blood pump and reducing the possibility of thrombosis.

The sensor 402 is disposed on the upper portion of the pump holder and is configured to sense the rotation of the rotator 302 in the pump housing 301 and provide sensed information to the control portion 403.

The control portion 403 generates control information through analysis and calculation according to the sensed information from the sensor, and controls the electromagnet 401 through a control circuit, so that the rotator 302 rotates without contact at all with the pump housing 301 under the combined action of the electromagnet 401, the driving permanent magnets 2022, and the first radial support permanent magnet 102.

The foregoing descriptions are merely a preferred embodiment of the present disclosure, but are not intended to limit the present disclosure. The present disclosure may include various modifications and changes for a person skilled in the art. Any modification, equivalent replacement, or improvement made within the spirit and principle of the present disclosure shall fall within the protection scope of the present disclosure.

The specific implementations of the present disclosure are described above with reference to the accompanying drawings, but are not intended to limit the protection scope of the present disclosure. A person skilled in the art should understand that various modifications or deformations may be made without creative efforts based on the technical solutions of the present disclosure, and such modifications or deformations shall fall within the protection scope of the present disclosure.

1. An extracorporeal circulation blood pump, characterized by comprising a pump holder, a pump body, a drive mechanism, and a control mechanism, wherein
the pump holder has an accommodating cavity for carrying the pump body, and a plurality of first radial support permanent magnets are provided on an inner edge of the accommodating cavity;
the pump body comprises a pump housing containing a rotator with a second radial support permanent magnet disposed on an outer circumference of a lower end in the rotator and a plurality of driven permanent magnets circumferentially distributed on the lower end in the rotator, and the first and second radial support permanent magnets are configured to interact with each other to generate a repulsive force, so that the rotator is rotatable in a radial direction without contact;
the drive mechanism, disposed at a bottom portion of the accommodating cavity, comprises a driving member and a rotating head, the driving member provides power to the blood pump through the rotating head, the rotating head comprises a rotating disc connected to an output end of the driving member and a plurality of driving permanent magnets circumferentially disposed on the rotating disc, and a coupling between the driving permanent magnets and the driven permanent magnets is achieved by magnetic attraction, so that the rotator is rotatable in an axial direction without contact; and
the control mechanism comprises an electromagnet disposed on an upper portion of the pump holder and a control portion, the electromagnet is provided to generate an axial upward attraction to the rotator in the pump, and a magnitude of the attraction is controlled by the control portion.

2. The extracorporeal circulation blood pump according to claim 1, wherein the pump holder comprises the upper portion and a lower portion that are movably connected to each other and is able to be opened and closed freely, and the first radial support permanent magnets are disposed on a lower portion of the pump holder.

3. The extracorporeal circulation blood pump according to claim 2, wherein the upper portion of the pump holder comprises a recess, an opening for accommodating the pump housing is provided in a middle portion of the recess, the recess is able to accommodate an upper surface of the pump body, and the electromagnet is circumferentially disposed at the opening on the upper portion of the pump holder.

4. The extracorporeal circulation blood pump according to claim 2, wherein the lower portion of the pump holder is provided with a stepped recess, the first radial support permanent magnets are disposed on one step of the stepped recess, and the drive mechanism is disposed at a bottom portion of the pump holder.

5. The extracorporeal circulation blood pump according to claim 2, wherein the upper portion and the lower portion of the pump holder are connected by a rotating shaft on one side and are provided with a fastener on the other side.

6. The extracorporeal circulation blood pump according to claim 1, wherein the drive mechanism comprises a driving motor and the rotating head, the driving motor is fixed on the pump holder, and the rotating head is fixed on a top portion of a rotating shaft of the driving motor, a middle portion of the rotating disc of the rotating head is connected to the rotating shaft of the driving motor, and the driving permanent magnets are embedded circumferentially on the rotating disc.

7. The extracorporeal circulation blood pump according to claim 6, wherein the rotator comprises a complex impeller and a permanent magnet assembly, the complex impeller is located on an upper portion of the rotator, and the permanent magnet assembly is disposed on a lower portion of the rotator.

8. The extracorporeal circulation blood pump according to claim 7, wherein the complex impeller comprises a magnetic conductor and an impeller body, the magnetic conductor is embedded in an upper cover of the impeller body, and the impeller body has a round hole in a center of the upper cover for the circulation of blood, and blood flows from an inlet of the pump housing, through the round hole and rotating blades, into a flow channel in the pump housing, and out of an outlet of the pump housing.

9. The extracorporeal circulation blood pump according to claim 7, wherein the permanent magnet assembly comprises the second radial support permanent magnet, the driven permanent magnets, and a sealed chamber, the sealed chamber is a cylindrical structure provided in a rotation center of the rotator and having a round hole, the second radial support permanent magnet is circumferentially embedded on an outer side of the sealed chamber, and the driven permanent magnets are circumferentially embedded on an inner side of the sealed chamber.

10. The extracorporeal circulation blood pump according to claim 1, wherein the control mechanism comprises the electromagnet, a sensor, and the control portion, and the sensor is disposed on the upper portion of the pump holder and is configured to sense a rotation of the rotator and provide sensed information to the control portion.

11. The extracorporeal circulation blood pump according to claim 10, wherein the control portion generates control information through analysis and calculation according to the sensed information from the sensor, and controls the electromagnet through a control circuit, so that the rotator rotates without contact at all with the pump housing under a combined action of the electromagnet, the driving permanent magnets, and the first and second radial support permanent magnets.

12. A method for operation of the extracorporeal circulation blood pump according to claim 1, the method comprising:
activating the drive mechanism to rotate the rotating head, thereby driving the rotator in the pump body to rotate;
controlling the electromagnet, resulting in the axial upward attraction from the electromagnet to the rotator, so that the attraction matches with a coupling attraction between the driving permanent magnets and the driven permanent magnets, and enabling the rotator to rotate in the axial direction without contact; and
configuring the first and second radial support permanent magnets to interact with each other to generate the repulsive force, enabling the rotator to rotate in the radial direction without contact, and leading to rotation without contact at all with the pump housing.
